SEQUENCE LISTING (3 sequences)

SEQ ID NO: 1 - protein, 5 aa, Artificial
  Synthetic peptide
CRGDC

SEQ ID NO: 2 - protein, 9 aa, Artificial
  Synthetic peptide
CXCRGDCXC

SEQ ID NO: 3 - protein, 9 aa, Artificial
  Synthetic peptide
CDCRGDCFC

(19) United States
(12) Patent Application Publication
(10) Pub. No.: US 2011/0123569 A1
(43) Pub. Date: May 26, 2011

(54) VACCINE DIRECTED AGAINST ADENOVIRUS SEROTYPE 14

(75) Inventors: Jason G.D. Gall, Germantown, MD (US); Christoph Kahl, Gaithersburg, MD (US); Gary J. Nabel, Bethesda, MD (US)

(73) Assignees: GenVec, Inc., Gaithersburg, MD (US); The Government of the United States of America, as represented by the Secretary, Rockville, MD (US)

(21) Appl. No.: 12/747,658

(22) PCT Filed: Dec. 11, 2008
(86) PCT No.: PCT/US08/86452

§ 371 Date: Dec. 27, 2010

Related U.S. Application Data
(60) Provisional application No. 61/013,202, filed on Dec. 12, 2007.

(51) Int. Cl. A61K 39/235; C12N 7/00; A61P 31/12

(52) U.S. Cl. 424/233.1; 435/235.1

(57) ABSTRACT

The invention is directed to a live attenuated serotype 14 adenovirus, and a method of inducing an immune response against a serotype 14 adenovirus in a mammal using the live attenuated serotype 14 adenovirus.

### STATEMENT REGARDING FEDERALLY SPONSORED RESEARCH AND DEVELOPMENT

This invention was made with Government support under Grant Number R43 AI062176-01 A2 awarded by the National Institutes of Health. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,137 Byte ASCII (Text) file named “704107_ST25.TXT,” created on Dec. 11, 2008.

### BACKGROUND OF THE INVENTION

Infection by adenovirus serotype 14 (Ad14) in humans has been rarely reported. Recently, Ad14 has become an emerging serotype of adenovirus that can cause severe and sometimes fatal respiratory illness in humans, including healthy young adults (see Centers for Disease Control and Prevention, Morb. Mortal. Wkly. Rep., 56(45): 1181-84 (2007), and Metzgar et al., J. Infectious Diseases, 196: 1465-73 (2007)). In May 2006, a 12-day old infant in New York died from respiratory illness caused by an Ad14 infection.

Since February 2007, an outbreak of cases of respiratory illness associated with adenovirus infection has been reported among basic military trainees at Lackland Air Force Base (LAFB) in Texas. Out of 423 respiratory specimens obtained from LAFB, 268 (63%) tested positive for adenovirus, 118 (44%) of the 268 were serotyped, and 106 (90%) of those serotyped were Ad14.

In May 2007, three residents of a residential-care facility in Washington State tested positive for Ad14. These patients required intensive care and mechanical ventilation for severe pneumonia. In early April 2007, 17 patients were reported to have been admitted at an Oregon hospital for severe pneumonia. Samples from 15 of these patients tested positive for Ad14. The Oregon Public Health Division later identified samples from 31 patients from November 2006 through April 2007 as positive for Ad14. Oregon reported a total of seven Ad14-related deaths. Other Ad14 outbreaks have been reported in South Carolina.

Fifty-three (38%) of the Ad14-positive patients described above were hospitalized, including 24 (17%) who were admitted to intensive care units (ICUs); nine (5%) patients died. Ad14 isolates from all four states were identical by sequence data from the full hexon and fiber genes. However, the isolates were distinct from the Ad14 reference strain (i.e., DeWitt strain) (Van Der Veen et al., Am. J. Hyg., 65: 119-129 (1957)), suggesting the emergence and spread of a new Ad14 variant in the United States (see Centers for Disease Control and Prevention, Morb. Mortal. Wkly. Rep., 56(45): 1181-84 (2007).

Thus, there is a need for a vaccine and immunization methods that effectively target emergent strains of Ad14. This invention provides such a method.

### BRIEF SUMMARY OF THE INVENTION

The invention provides a method of inducing an immune response against a serotype 14 adenovirus in a mammal. The method comprises administering to the mammal a live attenuated serotype 14 adenovirus, whereupon an immune response against a serotype 14 adenovirus is induced in the mammal.

The invention also provides a live attenuated serotype 14 adenovirus, as well as a composition (e.g., a vaccine) comprising such a live attenuated serotype 14 adenovirus.

### DETAILED DESCRIPTION OF THE INVENTION

The invention provides a method of inducing an immune response against a serotype 14 adenovirus in a mammal. Adenovirus is a medium-sized (90-100 nm), nonenveloped icosohedral virus containing 36 kb of double-stranded DNA. There are 49 immunologically distinct types of adenovirus that can cause human infections: subgroup A (e.g., serotypes 12, 18, and 31), subgroup B (e.g., serotypes 3, 7, 11, 14, 16, 21, 34, 35, and 50), subgroup C (e.g., serotypes 2 and 5) subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), and an unclassified subgroup (e.g., serotypes 49 and 51). Wild-type serotype 14 adenovirus has been deposited as GenBank Accession No. AY803294. A new circulating strain of serotype 14 adenovirus, 1968T, also has been described.

Adenoviruses most commonly cause respiratory illness, but also can cause various other illnesses, such as gastroenteritis, conjunctivitis, cystitis, and rash illness. Symptoms of respiratory illness caused by adenovirus infection range from the common cold syndrome to pneumonia, croup, and bronchitis. Patients with compromised immune systems are especially susceptible to severe complications of adenovirus infection. Acute respiratory disease (ARD), first recognized among military recruits during World War II, can be caused by adenovirus infections during conditions of crowding and stress.

The invention comprises administering to the mammal a live attenuated serotype 14 adenovirus. The live attenuated serotype 14 adenovirus can be produced in high titers and can efficiently be transferred to replicating and non-replicating cells. The live attenuated serotype 14 adenovirus remains epi-chromosomal, thereby eliminating the risks of random insertional mutagenesis and permanent alteration of the genotype of the target cell.

The term “live,” as used herein, refers to an adenovirus that retains the ability to enter cells and has not been physically inactivated by, for example, disruption (e.g., sonication), denaturing (e.g., using heat or solvents), or cross-linkage (e.g., via formalin cross-linking). The term “attenuated,” as used herein, refers to an adenovirus with reduced pathogenicity. Attenuation can be achieved by using a variety of methods known in the art. For example, serial passage of viruses in animals, eggs, or tissue culture can lead to the acquisition of a variety of mutations. Such mutations can result in reduced pathogenicity by, for example, preventing replication of the virus in mammalian (e.g., human cells), or by reducing, but not eliminating, replication capacity of the virus such that it can replicate in mammalian cells without inducing disease.

The inventive live attenuated serotype 14 adenovirus can require complementation of one or more regions of the adenoviral genome that are required for replication, as a result of, for example, a deficiency in at least one replication-essential gene function (i.e., such that the live attenuated serotype 14 adenovirus does not replicate in typical host cells, especially those in a mammal infected by the live attenuated serotype 14 adenovirus in the course of the inventive method). Such an adenovirus also is referred to in the art as a “replication-deficient” adenovirus. A deficiency in a gene, gene function, gene, or genomic region, as used herein, is defined as a mutation or deletion of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was mutated or deleted in whole or in part. Deletion of an entire gene region often is not required for disruption of a replication-essential gene function. However, if sufficient space in the adenoviral genome is needed for one or more transgenes, then the removal of a majority of a gene region may be desirable. While deletion of genetic material is preferred, mutation of genetic material by addition or substitution also is appropriate for disrupting gene function. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA-2).

The live attenuated serotype 14 adenovirus desirably requires complementation of, at most, the E1, E2A, and E4 regions of the adenoviral genome for propagation. Thus, for propagation, the live attenuated serotype 14 adenovirus can require complementation of, at most, (a) the E1 region, (b) the E2A region, (c) the E4 region, (d) the E1 and E2A regions, (e) the E1 and E4 regions, (f) the E2A and E4 regions, or (g) the E1, E2A, and E4 regions. Preferably, the live attenuated serotype 14 adenovirus requires complementation of, at most, the E1 and/or E4 regions of the adenoviral genome for propagation.

The live attenuated serotype 14 adenovirus can contain deletions and/or mutations in portions of the adenoviral genome other than the E1, E2A, and/or E4 regions. For example, the live attenuated serotype 14 adenovirus also can have deletions and/or mutations in the major late promoter (MLP), as discussed in International Patent Application Publication WO 00/00628, in the E3 region (e.g., an Xba I deletion of the E3 region), which does not include replication-essential gene functions, and/or in regions that include replication-essential gene functions but so as not to require complementation of regions other than E1, E2A, and/or E4 for propagation.

With respect to the E1 region, the live attenuated serotype 14 adenovirus can lack all or a portion of the E1A region and/or all or a portion of the E1B region, e.g., lack at least one replication-essential gene function of each of the E1A and E1B regions, thus requiring complementation of the E1A region and the E1B region of the adenoviral genome for replication. When the live attenuated serotype 14 adenovirus is E1-deficient, the adenoviral genome can comprise a deletion beginning at any nucleotide between nucleotides 465 to 500 (e.g., nucleotide 488) and ending at any nucleotide between nucleotides 2,900 to 2,950 (e.g., nucleotide 2,925) (based on the adenovirus serotype 14 genome (GenBank Accession No. AY803294). The endpoints defining the deleted nucleotide portions can be difficult to precisely determine and typically will not significantly affect the nature of the live attenuated serotype 14 adenovirus, i.e., each of the aforementioned nucleotide numbers can be +/−1, 2, 3, 4, 5, or even 10 or 20 nucleotides.

With respect to the E2A region, the live attenuated serotype 14 adenovirus preferably does not comprise a complete deletion of the E2A region, which deletion preferably is less than about 230 base pairs in length. Generally, the E2A region of the adenovirus codes for a DBP (DNA binding protein), which is a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196: 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that the live attenuated serotype 14 adenovirus contains this portion of the E2A region of the adenoviral genome. In particular, for example, the desired portion of the E2A region to be retained is that portion of the E2A region of the adenoviral genome which is defined by the 5′ end of the E2A region. This portion of the adenoviral genome desirably is included in the live attenuated serotype 14 adenovirus because it is not complemented in current E2A complementing cell lines so as to provide the desired level of viral propagation.

With respect to the E4 region, the live attenuated serotype 14 adenovirus can lack all or a portion of the E4 region. Desirably, the live attenuated serotype 14 adenovirus contains a deletion or mutation of Open Reading Frame (ORF) 6 of the E4 region, which is believed to be the only portion of the E4 region required for propagation of the live attenuated serotype 14 adenovirus.

In one embodiment of the invention, the live attenuated serotype 14 adenovirus comprises an adenoviral genome that lacks all or a portion of each of the E1 and E4 regions (i.e., the live attenuated serotype 14 adenovirus is an E1/E4-deficient adenovirus), preferably with the entire coding region of the E4 region having been deleted from the adenoviral genome. In other words, all the open reading frames (ORFs) of the E4 region have been removed. In another embodiment, the live attenuated serotype 14 adenovirus is rendered replication-deficient by deletion of all of the E1 region and by deletion of a portion of the E4 region. The E4 region of the live attenuated serotype 14 adenovirus can retain the native E4 promoter, polyadenylation sequence, and/or the right-side inverted terminal repeat (ITR).

In some embodiments, the live attenuated serotype 14 adenovirus which requires complementation of, for example, one or more gene functions of the E1 region and one or more gene functions of the E4 region can include a spacer sequence to provide viral growth in a complementing cell line similar to that achieved by the live attenuated serotype 14 adenovirus which requires complementation of one or more gene functions of only the E1 region. The spacer sequence can contain any nucleotide sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer element sequence can be coding or non-coding, native or non-native to adenovirus, native or non-native to serogroup B adenovirus, and native or non-native with respect to the adenoviral genome, i.e., serotype 14 adenovirus, but does not restore the replication-essential function to the deficient region. The spacer element can be located in any region of the live attenuated serotype 14 adenovirus, but preferably the spacer is located in the E1 region or E4 region of the adenoviral genome. The use of a spacer in an adenoviral vector is described in U.S. Pat. No. 5,851,806.

While the live attenuated serotype 14 adenovirus preferably requires complementation of, at most, replication-essential gene functions of the E1, E2A, and/or E4 regions of the adenoviral genome for replication (i.e., propagation), it is possible for the live attenuated serotype 14 adenovirus to have other deficiencies such that other complementation is required for propagation. In particular, the adenoviral genome can be modified to disrupt one or more replication-essential gene functions as desired by the practitioner, so long as the live attenuated serotype 14 adenovirus remains replication-deficient and can be propagated using, for example, complementing cells and/or exogenous DNA (e.g., helper adenovirus) encoding the disrupted replication-essential gene functions. In this respect, the live attenuated serotype 14 adenovirus can be deficient in replication-essential gene functions of only the early regions of the adenoviral genome, only the late regions of the adenoviral genome, both the early and late regions of the adenoviral genome, or all adenoviral genes (i.e., a high capacity adenovector (HC-Ad), see Morsy et al., Proc. Natl. Acad. Sci. USA, 95: 7876-7871 (1998), Chen et al., Proc. Natl. Acad. Sci USA, 94: 1645-1650 (1997), and Kochanek et al., Hum. Gene Ther., 10: 2451-2459 (1999)). The general preparation of replication-deficient adenovirus is disclosed in U.S. Pat. Nos. 5,837,511, 5,851,806, 6,127,175, 6,482,616, and 7,195,896; U.S. Patent Application Publications 2001/0043922 A1, 2002/0004040 A1, 2002/0110545 A1, and 2004/0161848 A1; and International Patent Application Publications WO 94/28152, WO 95/02697, WO 95/16772, WO 95/34671, WO 96/22378, WO 97/12986, WO 97/21826, and WO 03/022311.

The live attenuated serotype 14 adenovirus typically will be produced in a complementing cell line that provides gene functions not present in the live attenuated serotype 14 adenovirus, but required for viral propagation, at appropriate levels in order to generate high titers of live attenuated serotype 14 adenovirus stock. Desirably, the complementing cell line comprises, integrated into the cellular genome, adenoviral nucleic acid sequences which encode gene functions required for adenoviral propagation. A preferred cell line complements for at least one and preferably all replication-essential gene functions not present in a live attenuated adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the live attenuated serotype 14 adenovirus, which minimizes, and practically eliminates, the possibility of the adenoviral genome recombining with the cellular DNA. Accordingly, the presence of replication competent adenoviruses (RCA) is minimized if not avoided in the live attenuated serotype 14 adenovirus stock, which, therefore, is suitable for certain therapeutic purposes, especially vaccination purposes. The lack of RCA in the live attenuated serotype 14 adenovirus stock avoids the replication of serotype 14 adenovirus in non-complementing cells. Construction of such a complementing cell lines involve standard molecular biology and cell culture techniques, such as those described in Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Complementing cell lines for producing the live attenuated serotype 14 adenovirus include, but are not limited to, 293 cells (described in, e.g., Graham et al., J. Gen. Virol., 36, 59-72 (1977)), PER.C6 cells (described in, e.g., International Patent Application Publication WO 97/00326, and U.S. Pat. Nos. 5,994,128 and 6,033,908), and 293-ORF6 cells (described in, e.g., International Patent Application Publication WO 95/34671, U.S. Pat. No. 7,195,896, and Brough et al., J. Virol., 71: 9206-9213 (1997)). Additional complementing cells are described in, for example, U.S. Pat. Nos. 6,677,156 and 6,682,929, and International Patent Application Publication WO 03/20879. In some instances, the cellular genome need not comprise nucleic acid sequences, the gene products of which complement for all of the deficiencies of the live attenuated serotype 14 adenovirus. One or more replication-essential gene functions lacking in the live attenuated serotype 14 adenovirus can be supplied by a helper virus, e.g., a live attenuated serotype 14 adenovirus that supplies in trans one or more essential gene functions required for replication of the desired live attenuated serotype 14 adenovirus. Helper virus is often engineered to prevent packaging of infectious helper virus. For example, one or more replication-essential gene functions of the E1 region of the adenoviral genome are provided by the complementing cell, while one or more replication-essential gene functions of the E4 region of the adenoviral genome are provided by a helper virus.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate construction of the live attenuated serotype 14 adenovirus. For example, it can be advantageous to remove or introduce restriction enzyme sites in the adenoviral genome. Such benign mutations often have no detectable adverse effect on viral functioning.

Similarly, the coat protein of the live attenuated serotype 14 adenovirus can be manipulated to alter the binding specificity or recognition of the adenovirus for a receptor on a potential host cell. For adenovirus, such manipulations can include deletion of regions of adenovirus coat proteins (e.g., fiber, penton, or hexon), insertions of various native or non-native ligands into portions of a coat protein, and the like. Manipulation of the coat protein can broaden the range of cells infected by the live attenuated serotype 14 adenovirus or enable targeting of the live attenuated serotype 14 adenovirus to a specific cell type.

Any suitable technique for altering native binding to a host cell, such as native binding of the fiber protein to its cellular receptor, can be employed. For example, differing fiber lengths can be exploited to ablate native binding to cells. This optionally can be accomplished via the addition of a binding sequence to the penton base or fiber knob. This addition of a binding sequence can be done either directly or indirectly via a bispecific or multispecific binding sequence. In an alternative embodiment, the adenoviral fiber protein can be modified to reduce the number of amino acids in the fiber shaft, thereby creating a “short-shafted” fiber (as described in, for example, U.S. Pat. No. 5,962,311). Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of a live attenuated serotype 14 adenovirus comprising a short-shafted fiber enables targeting of the adenovirus to a desired cell-surface receptor by the introduction of a normative amino acid sequence either into the penton base or the fiber knob.

In yet another embodiment, the nucleic acid residues encoding amino acid residues associated with native substrate binding can be changed, supplemented, or deleted (see, e.g., International Patent Application Publication WO 00/15823, Einfeld et al., J. Virol., 75(23): 11284-11291 (2001), and van Beusechem et al., J. Virol., 76(6): 2753-2762 (2002)) such that the live attenuated serotype 14 adenovirus incorporating the mutated nucleic acid residues (or having the fiber protein encoded thereby) is less able to bind its native substrate. In this respect, the native cellular receptor for Ad14 has yet to be definitively determined. Recent studies suggest that the native cellular receptor for Ad14 is the CD46 cell surface protein (see, e.g., Sakurai et al., Current Gene Therapy, 7(4): 229-238 (2007), and Marttila et al., J. Virol., 79: 14429-14436 (2005)). In addition, there is evidence that Ad14 may bind to another as yet unidentified receptor in addition to CD46 (see Tuve et al., J. Virol., 80: 12109-12120 (2006)). In any event, the native cellular binding sites of the live attenuated serotype 14 adenovirus, such as the knob domain of the adenoviral fiber protein and an Arg-Gly-Asp (RGD) sequence located in the adenoviral penton base, respectively, can be removed or disrupted.

Any suitable amino acid residue(s) of a fiber protein that mediates or assists in the interaction between the knob and the native cellular receptor can be mutated or removed, so long as the fiber protein is able to trimerize. Similarly, amino acids can be added to the fiber knob as long as the fiber protein retains the ability to trimerize. Suitable residues include amino acids within the exposed loops of the fiber knob domain, such as, for example, the AB loop, the DE loop, the FG loop, and the HI loop.

Any suitable amino acid residue(s) of a penton base protein that mediates or assists in the interaction between the penton base and integrins can be mutated or removed. Suitable residues include, for example, an RGD amino acid sequence motif located in the hypervariable region of the Ad14 penton base protein. The native integrin binding sites on the penton base protein also can be disrupted by modifying the nucleic acid sequence encoding the native RGD motif such that the native RGD amino acid sequence is conformationally inaccessible for binding to an integrin receptor, such as by inserting a DNA sequence into or adjacent to the nucleic acid sequence encoding the adenoviral penton base protein.

The live attenuated serotype 14 adenovirus can comprise a fiber protein and a penton base protein that do not bind to their respective native cellular binding sites. Alternatively, the live attenuated serotype 14 adenovirus comprises fiber protein and a penton base protein that bind to their respective native cellular binding sites, but with less affinity than the corresponding wild-type coat proteins. The live attenuated serotype 14 adenovirus exhibits reduced binding to native cellular binding sites if a modified adenoviral fiber protein and penton base protein binds to their respective native cellular binding sites with at least about 5-fold, 10-fold, 20-fold, 30-fold, 50-fold, or 100-fold less affinity than a non-modified adenoviral fiber protein and penton base protein of the same serotype.

The live attenuated serotype 14 adenovirus also can comprise a chimeric coat protein comprising a non-native amino acid sequence that binds a substrate (i.e., a ligand), such as a cellular receptor other than a native cellular receptor. The non-native amino acid sequence of the chimeric adenoviral coat protein allows the live attenuated serotype 14 adenovirus comprising the chimeric coat protein to bind and, desirably, infect host cells not naturally infected by a corresponding adenovirus without the non-native amino acid sequence (i.e., host cells not infected by the corresponding wild-type adenovirus), to bind to host cells naturally infected by the corresponding wild-type adenovirus with greater affinity than the corresponding adenovirus without the non-native amino acid sequence, or to bind to particular target cells with greater affinity than non-target cells. A “non-native” amino acid sequence can comprise an amino acid sequence not naturally present in the adenoviral coat protein or an amino acid sequence found in the adenoviral coat but located in a non-native position within the capsid. By “preferentially binds” is meant that the non-native amino acid sequence binds a receptor, such as, for instance, αvβ3 integrin, with at least about 3-fold greater affinity (e.g., at least about 5-fold, 10-fold, 15-fold, 20-fold, 25-fold, 35-fold, 45-fold, or 50-fold greater affinity) than the non-native ligand binds a different receptor, such as, for instance, αvβ1 integrin.

The live attenuated serotype 14 adenovirus can comprise a chimeric coat protein comprising a non-native amino acid sequence that confers to the chimeric coat protein the ability to bind to an immune cell more efficiently than a wild-type adenoviral coat protein. In particular, the live attenuated serotype 14 adenovirus can comprise a chimeric adenoviral fiber protein comprising a non-native amino acid sequence which facilitates uptake of the live attenuated serotype 14 adenovirus by immune cells, preferably antigen presenting cells, such as dendritic cells, monocytes, and macrophages. In a preferred embodiment, the live attenuated serotype 14 adenovirus comprises a chimeric fiber protein comprising an amino acid sequence (e.g., a non-native amino acid sequence) comprising an RGD motif including, but not limited to, CRGDC (SEQ ID NO: 1), CXCRGDCXC (SEQ ID NO: 2), wherein X represents any amino acid, and CDCRGDCFC (SEQ ID NO: 3), which increases transduction efficiency of the live attenuated serotype 14 adenovirus into dendritic cells. The RGD-motif, or any non-native amino acid sequence, preferably is inserted into the adenoviral fiber knob region, ideally in an exposed loop of the adenoviral knob, such as the HI loop. A non-native amino acid sequence also can be appended to the C-terminus of the adenoviral fiber protein, optionally via a spacer sequence. The spacer sequence preferably comprises between one and two-hundred amino acids, and can (but need not) have an intended function.

The non-native amino acid sequence can optionally recognize a protein typically found on dendritic cell surfaces such as adhesion proteins, chemokine receptors, complement receptors, co-stimulation proteins, cytokine receptors, high level antigen presenting molecules, homing proteins, marker proteins, receptors for antigen uptake, signaling proteins, virus receptors, etc. Examples of such potential ligand-binding sites in dendritic cells include αvβ3 integrins, αvβ5 integrins, 2A1, 7-TM receptors, CD1, CD11a, CD11b, CD11c, CD21, CD24, CD32, CD4, CD40, CD44 variants, CD46, CD49d, CD50, CD54, CD58, CD64, ASGPR, CD80, CD83, CD86, E-cadherin, integrins, M342, MHC-I, MHC-II, MIDC-8, MMR, OX62, p200-MR6, p55, S100, TNF-R, etc. Where dendritic cells are targeted, the non-native amino acid sequence preferably recognizes the CD40 cell surface protein, such as, for example, by way of a CD-40 (bi)specific antibody fragment or by way of a domain derived from the CD40L polypeptide.

The non-native amino acid sequence optionally can recognize a protein typically found on macrophage cell surfaces, such as phosphatidylserine receptors, vitronectin receptors, integrins, adhesion receptors, receptors involved in signal transduction and/or inflammation, markers, receptors for induction of cytokines, or receptors up-regulated upon challenge by pathogens, members of the group B scavenger receptor cysteine-rich (SRCR) superfamily, sialic acid binding receptors, members of the Fc receptor family, B7-1 and B7-2 surface molecules, lymphocyte receptors, leukocyte receptors, antigen presenting molecules, and the like. Examples of suitable macrophage surface target proteins include, but are not limited to, heparin sulfate proteoglycans, αvβ3 integrins, αvβ5 integrins, B7-1, B7-2, CD11c, CD13, CD16, CD163, CD1a, CD22, CD23, CD29, Cd32, CD33, CD36, CD44, CD45, CD49e, CD52, CD53, CD54, CD71, CD87, CD9, CD98, Ig receptors, Fc receptor proteins (e.g., subtypes of Fcα, Fcγ, Fcε, etc.), folate receptor b, HLA Class I, Sialoadhesin, siglec-5, and the toll-like receptor-2 (TLR2).

The non-native amino acid sequence can recognize a protein typically found on B-cell surfaces, such as integrins and other adhesion molecules, complement receptors, interleukin receptors, phagocyte receptors, immunoglobulin receptors, activation markers, transferrin receptors, members of the scavenger receptor cysteine-rich (SRCR) superfamily, growth factor receptors, selectins, MHC molecules, TNF-receptors, and TNF-R associated factors. Examples of typical B-cell surface proteins include β-glycan, B cell antigen receptor (BAC), B7-2, B-cell receptor (BCR), C3d receptor, CD1, CD18, CD19, CD20, CD21, CD22, CD23, CD35, CD40, CD5, CD6, CD69, CD69, CD71, CD79a/CD79b dimer, CD95, endoglin, Fas antigen, human Ig receptors, Fc receptor proteins (e.g., subtypes of Fca, Fcg, Fcε, etc.), IgM, gp200-MR6, Growth Hormone Receptor (GH-R), ICAM-1, ILT2, CD85, MHC class I and II molecules, transforming growth factor receptor (TGF-R), α4β7 integrin, and αvβ3 integrin.

In another embodiment, the live attenuated serotype 14 adenovirus can comprise a chimeric virus coat protein that is not selective for a specific type of eukaryotic cell. The chimeric coat protein differs from a wild-type coat protein by an insertion of a non-native amino acid sequence into or in place of an internal coat protein sequence, or attachment of a non-native amino acid sequence to the N- or C-terminus of the coat protein. For example, a ligand comprising about five to about nine lysine residues (preferably seven lysine residues) is attached to the C-terminus of the adenoviral fiber protein via a non-functional spacer sequence. In this embodiment, the chimeric virus coat protein efficiently binds to a broader range of eukaryotic cells than a wild-type virus coat, such as described in U.S. Pat. No. 6,465,253 and International Patent Application Publication WO 97/20051.

The ability of the live attenuated serotype 14 adenovirus to recognize a potential host cell can be modulated without genetic manipulation of the coat protein, i.e., through use of a bi-specific molecule. For instance, complexing an adenovirus with a bispecific molecule comprising a penton base-binding domain and a domain that selectively binds a particular cell surface binding site enables the targeting of the live attenuated serotype 14 adenovirus to a particular cell type. Likewise, an antigen can be conjugated to the surface of the adenoviral particle through non-genetic means.

A non-native amino acid sequence can be conjugated to any of the adenoviral coat proteins to form a chimeric adenoviral coat protein. Therefore, for example, a non-native amino acid sequence can be conjugated to, inserted into, or attached to a fiber protein, a penton base protein, a hexon protein, proteins IX, VI, or IIIa, etc. Methods for employing such proteins are well known in the art (see, e.g., U.S. Pat. Nos. 5,543,328; 5,559,099; 5,712,136; 5,731,190; 5,756,086; 5,770,442; 5,846,782; 5,962,311; 5,965,541; 5,846,782; 6,057,155; 6,127,525; 6,153,435; 6,329,190; 6,455,314; 6,465,253; 6,576,456; 6,649,407; 6,740,525, 6,951,755, and International Patent Application Publications WO 96/07734, WO 96/26281, WO 97/20051, WO 98/07877, WO 98/07865, WO 98/40509, WO 98/54346, WO 00/15823, WO 01/58940, and WO 01/92549). The chimeric adenoviral coat protein can be generated using standard recombinant DNA techniques known in the art. Preferably, the nucleic acid sequence encoding the chimeric adenoviral coat protein is located within the adenoviral genome and is operably linked to a promoter that regulates expression of the coat protein in a wild-type adenovirus. Alternatively, the nucleic acid sequence encoding the chimeric adenoviral coat protein is located within the adenoviral genome and is part of an expression cassette which comprises genetic elements required for efficient expression of the chimeric coat protein.

The coat protein portion of the chimeric adenovirus coat protein can be a full-length adenoviral coat protein to which the non-native amino acid sequence is appended, or it can be truncated, e.g., internally or at the C- and/or N-terminus. However modified (including the presence of the non-native amino acid), the chimeric coat protein preferably is able to incorporate into an adenoviral capsid. Where the non-native amino acid sequence is attached to the fiber protein, preferably it does not disturb the interaction between viral proteins or fiber monomers. Thus, the non-native amino acid sequence preferably is not itself an oligomerization domain, as such can adversely interact with the trimerization domain of the adenovirus fiber. Preferably the non-native amino acid sequence is added to the virion protein, and is incorporated in such a manner as to be readily exposed to a substrate, cell surface-receptor, or immune cell (e.g., at the N- or C-terminus of the adenoviral protein, attached to a residue facing a substrate, positioned on a peptide spacer, etc.) to maximally expose the non-native amino acid sequence. Ideally, the non-native amino acid sequence is incorporated into an adenoviral fiber protein at the C-terminus of the fiber protein (and attached via a spacer) or incorporated into an exposed loop (e.g., the HI loop) of the fiber to create a chimeric coat protein. Where the non-native amino acid sequence is attached to or replaces a portion of the penton base, preferably it is within the hypervariable regions to ensure that it contacts the substrate, cell surface receptor, or immune cell. Where the non-native amino acid sequence is attached to the hexon, preferably it is within a hypervariable region (Crawford-Miksza et al., J. Virol., 70(3): 1836-44 (1996)). Where the non-native amino acid is attached to or replaces a portion of pIX, preferably it is within the C-terminus of pIX. Use of a spacer sequence to extend the non-native amino acid sequence away from the surface of the adenoviral particle can be advantageous in that the non-native amino acid sequence can be more available for binding to a receptor, and any steric interactions between the non-native amino acid sequence and the adenoviral fiber monomers can be reduced.

Binding affinity of a non-native amino acid sequence to a cellular receptor can be determined by any suitable assay, a variety of which assays are known and are useful in selecting a non-native amino acid sequence for incorporating into an adenoviral coat protein. Desirably, the transduction levels of host cells are utilized in determining relative binding efficiency. Thus, for example, host cells displaying αvβ3 integrin on the cell surface (e.g., MDAMB435 cells) can be exposed to a live attenuated serotype 14 adenovirus comprising the chimeric coat protein and the corresponding adenovirus without the non-native amino acid sequence, and then transduction efficiencies can be compared to determine relative binding affinity. Similarly, both host cells displaying αvβ3 integrin on the cell surface (e.g., MDAMB435 cells) and host cells displaying predominantly αvβ1 on the cell surface (e.g., 293 cells) can be exposed to the live attenuated serotype 14 adenovirus comprising the chimeric coat protein, and then transduction efficiencies can be compared to determine binding affinity.

In other embodiments (e.g., to facilitate purification or propagation within a specific engineered cell type), a non-native amino acid (e.g., ligand) can bind a compound other than a cell-surface protein. Thus, the ligand can bind blood- and/or lymph-borne proteins (e.g., albumin), synthetic peptide sequences such as polyamino acids (e.g., polylysine, polyhistidine, etc.), artificial peptide sequences (e.g., FLAG), and RGD peptide fragments (Pasqualini et al., J. Cell. Biol., 130: 1189 (1995)). A ligand can even bind non-peptide substrates, such as plastic (e.g., Adey et al., Gene, 156: 27 (1995)), biotin (Saggio et al., Biochem. J., 293: 613 (1993)), a DNA sequence (Cheng et al., Gene, 171: 1 (1996), and Krook et al., Biochem. Biophys., Res. Commun., 204: 849 (1994)), streptavidin (Geibel et al., Biochemistry, 34: 15430 (1995), and Katz, Biochemistry, 34: 15421 (1995)), nitrostreptavidin (Balass et al., Anal. Biochem., 243: 264 (1996)), heparin (Wickham et al., Nature Biotechnol., 14: 1570-73 (1996)), and other substrates.

Modifications to adenoviruses are described in U.S. Pat. Nos. 5,543,328; 5,559,099; 5,712,136; 5,731,190; 5,756,086; 5,770,442; 5,846,782; 5,871,727; 5,885,808; 5,922,315; 5,962,311; 5,965,541; 6,057,155; 6,127,525; 6,153,435; 6,329,190; 6,455,314; 6,465,253; 6,576,456; 6,649,407; 6,740,525, 6,951,755, and 7,195,896; U.S. Patent Application Publication 2003/0099619 A1, and International Patent Applications WO 96/07734, WO 96/26281, WO 97/20051, WO 98/07865, WO 98/07877, WO 98/40509, WO 98/54346, WO 00/15823, WO 01/58940, and WO 01/92549.

Typically, by removing all or part of the adenoviral genome, the resulting live attenuated serotype 14 adenovirus is able to accept inserts of heterologous nucleic acid sequences while retaining the ability to be packaged into adenoviral capsids. In a preferred embodiment, however, the live attenuated serotype 14 adenovirus does not comprise a heterologous nucleic acid sequence. Thus, the live attenuated serotype 14 adenovirus preferably comprises a deletion of all or part of the E1, E2A, and/or E4 regions, as well as optionally the E3 region, of the adenoviral genome, but does not contain a heterologous nucleic acid sequence inserted into any of the deleted regions of the adenoviral genome.

However, in other embodiments it may be appropriate to insert one or more heterologous nucleic acid sequences into one or more regions deleted from the adenovirus. In this respect, a heterologous nucleic acid sequence can be positioned in the E1 region, the E2A region, and/or the E4 region, as well as optionally the E3 region, of the adenoviral genome. Indeed, a heterologous nucleic acid sequence can be inserted anywhere in the adenoviral genome so long as the position does not prevent expression of the heterologous nucleic acid sequence or interfere with packaging of the live attenuates serotype 14 adenovirus. Any type of nucleic acid sequence (e.g., DNA, RNA, and cDNA) that can be inserted into an adenovirus can be used in connection with the invention. Preferably, the heterologous nucleic acid sequence is DNA, and preferably encodes a protein (i.e., one or more nucleic acid sequences encoding one or more proteins).

The heterologous nucleic acid sequence can encode an antigen. An “antigen” is a molecule that induces an immune response in a mammal. An “immune response” can entail, for example, antibody production and/or the activation of immune effector cells (e.g., T cells). An antigen in the context of the invention can comprise any subunit, fragment, or epitope of any proteinaceous molecule, including a protein or peptide of viral, bacterial, parasitic, fungal, protozoan, prion, cellular, or extracellular origin, which ideally provokes an immune response in mammal, preferably leading to protective immunity. By “epitope” is meant a sequence on an antigen that is recognized by an antibody or an antigen receptor. Epitopes also are referred to in the art as “antigenic determinants.”

The heterologous nucleic acid sequence can encode an immune system stimulator to enhance or modify the immune response elicited by the live attenuated serotype 14 adenovirus. Examples of immune system stimulators include cytokines, lipopolysaccharide, double-stranded RNA, toll-like receptors (TLRs), and complement proteins (e.g., CD46). Preferably, the heterologous nucleic acid sequence encodes a cytokine. “Cytokines” are known in the art as non-antibody proteins secreted by specific cells (e.g., inflammatory leukocytes and some non-leukocytic cells), which act as intercellular mediators, such as by regulating immunity, inflammation, and hematopoiesis. Cytokines generally act locally in a paracrine or autocrine rather than endocrine manner. Cytokines can be classified as a lymphokine (cytokines made by lymphocytes), a monokine (cytokines made by monocytes), a chemokine (cytokines with chemotactic activities), and an interleukin (cytokines made by one leukocyte and acting on other leukocytes). The cytokine can be any suitable cytokine known in the art, including, but not limited to, interferons (e.g., IFN-alpha, IFN-beta, IFN-delta, IFN-omega, IFN-tau, and IFN-gamma), interleukins, RANTES, MCP-1, MIP-1α, and MIP-1β, granulocyte monocyte colony-stimulating factor (GM-CSF), and tumor necrosis factor (TNF) alpha.

When the live attenuated serotype 14 adenovirus comprises one or more heterologous nucleic acid sequences, each heterologous nucleic acid sequence desirably is operably linked to (i.e., under the transcriptional control of) one or more promoter and/or enhancer elements, for example, as part of a promoter-variable expression cassette. Techniques for operably linking sequences together are well known in the art. Any promoter or enhancer sequence can be used in the context of the invention, so long as sufficient expression of the heterologous nucleic acid sequence is achieved. Preferably, the promoter is a heterologous promoter, in that the promoter is not obtained from, derived from, or based upon a naturally occurring promoter of the live attenuated serotype 14 adenovirus. In this regard, the promoter can be, for example, a viral promoter or a cellular promoter. Moreover, the promoter can be constitutive, inducible (e.g., radiation-, light-, chemotherapy-, or drug-inducible), or tissue-specific. Suitable promoters are known in the art (see, e.g., International Patent Application Publication WO 2007/027860).

In the method of the invention, the live attenuated serotype 14 adenovirus preferably is administered to a mammal (e.g., a human), wherein the serotype 14 adenovirus particle induces an immune response. The live attenuated serotype 14 adenovirus can be administered to any mammal that can be infected by a serotype 14 adenovirus. Preferably, the live attenuated serotype 14 adenovirus is administered to a human. The immune response can be a humoral immune response, a cell-mediated immune response, or, desirably, a combination of humoral and cell-mediated immunity. Ideally, the immune response provides protection upon subsequent challenge with the serotype 14 adenovirus. However, protective immunity is not required in the context of the invention. The inventive method further can be used for antibody production and harvesting.

Administering the live attenuated serotype 14 adenovirus can be one component of a multistep regimen for inducing an immune response in a mammal. In particular, the inventive method can represent one arm of a prime and boost immunization regimen. The inventive method, therefore, can comprise administering to the mammal (a) a priming composition prior to administering the live attenuated serotype 14 adenovirus and/or (b) a boosting composition after administering the live attenuated serotype 14 adenovirus.

The priming and boosting compositions can comprise any suitable antigen as described herein (e.g. an inactivated virus, a protein, a peptide, or an epitope sequence). Preferably, the priming and/or boosting composition comprises a gene transfer vector. Any gene transfer vector can be employed in the priming composition or the boosting gene composition, including, but not limited to, a plasmid, a retrovirus, an adeno-associated virus, a vaccinia virus, a herpesvirus, an alphavirus, or an adenovirus. Ideally, the gene transfer vector is a plasmid, an alphavirus, or an adenoviral vector. The priming composition and the boosting composition can comprise the inventive live attenuated serotype 14 adenovirus, or a gene transfer vector that is different from the live attenuated serotype 14 adenovirus. Preferably, the priming composition and the boosting composition each comprise the inventive live attenuated serotype 14 adenovirus. In other words, the inventive method desirably involves multiple administrations of the live attenuated serotype 14 adenovirus. The priming composition and/or the boosting composition can be provided in any suitable timeframe (e.g., at least about 1 week, 2 weeks, 4 weeks, 8 weeks, 12 weeks, 16 weeks, or more prior to or following administration of the live attenuated serotype 14 adenovirus) to maintain immunity. In addition, the priming composition and/or the boosting composition can be administered multiple times during the course of a particular immunization regimen. For example, the immunization regimen can comprise two or more (e.g., 2, 3, 5, or more) administrations of a priming composition, followed by a single administration of the live attenuated serotype 14 adenovirus, followed by two or more (e.g., 2, 3, 5, or more) administrations of a boosting composition. Of course, in some cases administration of the inventive live attenuated serotype 14 adenovirus may be sufficient to induce a robust and protective immune response. In this regard, a priming and/or boosting regimen may not be necessary.

To maximize the effect of the priming/boosting regimen, the priming composition and/or the boosting composition can comprise a gene transfer vector comprising a heterologous nucleic acid sequence encoding an antigen. Alternatively, an immune response can be primed or boosted by administration of an antigen itself, e.g., an antigenic protein, inactivated pathogen, and the like. For example, an immune response can be primed and/or boosted by administration of an inactivated (e.g., heat-killed or chemically inactivated) attenuated serotype 14 adenovirus, or an inactivated wild-type serotype 14 adenovirus.

Any route of administration can be used to deliver the live attenuated serotype 14 adenovirus to the mammal. Indeed, although more than one route can be used to administer the live attenuated serotype 14 adenovirus, a particular route can provide a more immediate and more effective reaction than another route. Preferably, the live attenuated serotype 14 adenovirus is administered via intramuscular injection. A dose of live attenuated serotype 14 adenovirus also can be applied or instilled into body cavities, absorbed through the skin (e.g., via a transdermal patch), inhaled, ingested, topically applied to tissue, or administered parenterally via, for instance, intravenous, peritoneal, or intraarterial administration. The live attenuated serotype 14 adenovirus also can be administered in vivo by particle bombardment (e.g., a gene gun).

The dose of live attenuated serotype 14 adenovirus administered to the mammal will depend on a number of factors, including the size of a target tissue, the extent of any side-effects, the particular route of administration, and the like. The dose ideally comprises an “effective amount” of live attenuated serotype 14 adenovirus, i.e., a dose of live attenuated serotype 14 adenovirus which provokes a desired immune response in the mammal. The desired immune response can entail production of antibodies, protection upon subsequent challenge, immune tolerance, immune cell activation, and the like. Desirably, a single dose of the live attenuated serotype 14 adenovirus comprises at least about 1×10⁵ particles (which also is referred to as particle units) of the live attenuated serotype 14 adenovirus. The dose preferably is at least about 1×10⁶ particles (e.g., about 1×10⁶-1×10¹² particles), more preferably at least about 1×10⁷ particles, more preferably at least about 1×10⁸ particles (e.g., about 1×10⁸-1×10¹¹ particles), and most preferably at least about 1×10⁹ particles (e.g., about 1×10⁹-1×10¹⁰ particles) of the live attenuated serotype 14 adenovirus. The dose desirably comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles). In other words, a single dose of live attenuated serotype 14 adenovirus can comprise, for example, about 1×10⁶ particle units (pu), 2×10⁶ pu, 4×10⁶ pu, 1×10⁷ pu, 2×10⁷ pu, 4×10⁷ pu, 1×10⁸ pu, 2×10⁸ pu, 4×10⁸ pu, 1×10⁹ pu, 2×10⁹ pu, 4×10⁹ pu, 1×10¹⁰ pu, 2×10¹⁰ pu, 4×10¹⁰ pu, 1×10¹¹ pu, 2×10¹¹ pu, 4×10¹¹ pu, 1×10¹² pu, 2×10¹² pu, or 4×10¹² pu of the live attenuated serotype 14 adenovirus.

The live attenuated serotype 14 adenovirus desirably is administered in a composition, preferably a pharmaceutically acceptable (e.g., physiologically acceptable) composition, which comprises a carrier, preferably a pharmaceutically acceptable (e.g., physiologically acceptable) carrier and the live attenuated serotype 14 adenovirus. Any suitable carrier can be used within the context of the invention, and such carriers are well known in the art. The choice of carrier will be determined, in part, by the particular site to which the composition is to be administered and the particular method used to administer the composition. Ideally, the composition preferably is free of replication-competent adenovirus. The composition optionally can be sterile or sterile with the exception of the inventive live attenuated serotype 14 adenovirus.

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the carrier is a buffered saline solution. More preferably, the live attenuated serotype 14 adenovirus for use in the inventive method is administered in a composition formulated to protect the live attenuated serotype 14 adenovirus from damage prior to administration. For example, the composition can be formulated to reduce loss of the adenovirus on devices used to prepare, store, or administer the adenovirus, such as glassware, syringes, or needles. The composition can be formulated to decrease the light sensitivity and/or temperature sensitivity of the live attenuated serotype 14 adenovirus. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the live attenuated serotype 14 adenovirus, facilitate administration, and increase the efficiency of the inventive method. Formulations for adenovirus-containing compositions are further described in, for example, U.S. Pat. No. 6,225,289, U.S. Pat. No. 6,514,943, and International Patent Application Publication WO 00/34444.

The live attenuated serotype 14 adenovirus can be formulated for oral administration. Formulations suitable for oral administration include (a) liquid solutions, such as an effective amount of the live attenuated serotype 14 adenovirus dissolved in diluents, such as water, saline, or orange juice, (b) capsules, sachets or tablets, each containing a predetermined amount of the live attenuated serotype 14 adenovirus, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the live attenuated serotype 14 adenovirus in a flavor, usually sucrose and acacia or tragacanth, as well as pastilles comprising the live attenuated serotype 14 adenovirus in an inert base, such as gelatin and glycerin, or sucrose and acacia, emulsions, gels, and the like containing, in addition to the live attenuated serotype 14 adenovirus, such excipients as are known in the art.

The live attenuated serotype 14 adenovirus, alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non pressured preparations, such as in a nebulizer or an atomizer.

The live attenuated serotype 14 adenovirus can be formulated for topical administration. Topical formulations include for example, ointments, lotions, creams, gels, drops, suppositories, sprays, liquids, and powders. Conventional pharmaceutical carriers, aqueous, powder, oily bases, thickeners, and the like may be necessary or desirable.

The live attenuated serotype 14 adenovirus can be administered in or on a device that allows controlled or sustained release, such as a sponge, biocompatible meshwork, mechanical reservoir, or mechanical implant. Implants (see, e.g., U.S. Pat. No. 5,443,505) and devices (see, e.g., U.S. Pat. No. 4,863,457), such as an implantable device, e.g., a mechanical reservoir or an implant or a device comprised of a polymeric composition, can be particularly useful for administration of the live attenuated serotype 14 adenovirus. The live attenuated serotype 14 adenovirus also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The composition also can be formulated to enhance transduction efficiency. In addition, one of ordinary skill in the art will appreciate that the live attenuated serotype 14 adenovirus can be present in a composition with other therapeutic or biologically-active agents. For example, factors that control inflammation, such as ibuprofen or steroids, can be part of the composition to reduce swelling and inflammation associated with in vivo administration of the live attenuated serotype 14 adenovirus. Moreover, immune system stimulators or adjuvants, e.g., interleukins, lipopolysaccharide, and double-stranded RNA, can be administered to enhance or modify any immune response to the live attenuated serotype 14 adenovirus. Antibiotics, i.e., microbicides and fungicides, can be present to treat existing infection and/or reduce the risk of future infection.

The following examples further illustrate the invention but, of course, should not be construed as in any way limiting its scope.

### Example 1

This example demonstrates the construction and characterization of a live attenuated serotype 14 adenovirus.

Live attenuated serotype 14 adenovirus constructs will be generated from whole genome plasmids using a bacterial recombination system for adenovirus vector assembly (Campos et al., Hum. Gene Ther., 15(11): 1125-1130 (2004)). Briefly, the system utilizes homologous recombination between a pair of plasmids, i.e., a shuttle and base plasmid, in E. coli. The shuttle plasmid contains a deletion of the E1 region flanked by adenovirus sequences proximal to the E1 region. The base plasmid contains the entire adenovirus vector genome with a selection marker in the desired location for the deletion. Recombination occurs between the homologous sequences in the shuttle and base plasmid. The desired recombinant plasmid will consist of the adenoviral genome containing the E1 deletion.

The recombination-competent (Rec+) E. coli strain BJDE3 will be transformed with a mixture of the shuttle and base plasmids that have been linearized by restriction enzyme digestion and plated on selection medium. The desired recombinant clones will then be identified by standard plasmid DNA extraction, restriction enzyme analysis, and sequencing. The resultant plasmid, called pAd14nr, will be digested with Pme I to liberate the recombinant Ad14nr genome (rAd14nr) from the plasmid backbone, and 293-ORF6 cells will be transfected. Following transfection of the plasmid, the resultant adenovirus will be amplified by serial passaging and purification over cesium chloride gradients.

The purified adenovirus will be characterized using several assays. Specifically, the infectious and total particle titers will be determined, the genetic stability/integrity of rAd14nr will be confirmed by PCR analysis and DNA sequencing, and each stock will be tested for the presence of replication-competent adenovirus (RCA).

The yield of Ad14nr will be assessed in three successive expansions to 10 liter culture. Ad yields are expected to be at least 1×10¹³ particle units (pu) per liter. Genetic Structural Integrity (GSI) will be tested during 10 successive passages of Ad14nr to assure that virus stocks can be expanded to eventual commercial sized bioreactors. Using primer oligonucleotides flanking the deleted E1 region, PCR will be employed to probe for any changes occurring in the E1 region of Ad14nr. Stability of Ad14nr will be tested at routine storage temperatures. Stability of Ad5-based vectors has been demonstrated over five years at −20° C. and for over six months at 4° C. Similar stability has been observed for Ad35 vectors. Stability of Ad14nr stocks will be tested for at least three months at −20° C., 4° C., and 25° C. Time points tested will be 1, 7, and 14 days and 4, 8, and 12 weeks. Stability testing will be conducted using plaque forming assays and particle determinations.

This example demonstrates a method of producing and characterizing a live attenuated serotype 14 adenovirus.

### Example 2

This example demonstrates a method for measuring an immune response against a live attenuated serotype 14 adenovirus in a mammal.

The immunogenicity of Ad14nr will be tested in a mouse model and primate model. Specifically, Ad14nr will be prepared as described in Example 1 and purified. 10 Balb/c mice will be assigned to each of three dosage groups (i.e., 1×10⁸ pu, 1×10⁹ pu, and 1×10¹⁰ pu). An E1-deleted serotype 14 adenovirus comprising an expression cassette for a fragment of HIV envelope protein gp140B (Ad14gp140B) will serve as a positive control, while an E1-deleted Ad5 vector will serve as a negative control. Mice will be injected at day 0 with the appropriate adenovirus, and the neutralizing antibody titer will be assayed at specific time points (i.e., 0, 1, 3, 7, 14, and 21 days).

Primate immunogenicity testing will focus on confirming that immune responses are robust in primates at doses that are known to be immunogenic and well tolerated in humans for Ad5-based vaccines. In this regard, two cynomolgous monkeys will be assigned to each of two dosage groups (1×10¹⁰ pu and 1×10¹¹ pu). Monkeys will be injected at day 0 with Ad14nr, and the neutralizing antibody titer will be assayed at specific time points (i.e., 0, 1, 3, 7, 14, and 21 days).

Neutralizing antibody titer assays used to evaluate the presence of antibodies in infected military recruits (Morb. Mortal. Wkly. Rep., 56(45): 1181-1184 (2007)) will be used in the above-described immunogenicity experiments. A quantitative serum colorimetric micro-neutralization (SN) test will also be used, which was originally developed and validated for adenoviruses types 4 and 7 for the evaluation of a live oral vaccine (Lyons et al., Vaccine, 26(23): 2890-2898 (2008)). Prior to testing, each serum specimen will be inactivated at 56±2° C. for 30±2 min. The serum specimens will be tested in groups of 6 wells/dilution using 2-fold dilutions to cover the necessary range. Negative and positive reference sera will be tested at the same time. The challenge virus dose will be titrated in each assay in one-half log doses using 6 wells/dose. Each well will contain 100 tissue culture infectious dose TCID₅₀ (±0.7 log 10) of adenovirus of the appropriate serotype. After incubation for 1 hour, 100 μl containing 20,000±2,000 A549 cells/ml will be added. After 7 days, cells will be stained by incubation with neutral red solution, machine washed, and subsequently fixed using acid-alcohol. Plates will be read with the plate blank subtracted at 550 nm. Statistics will be conducted using ANOVA with Bonferonni correction for multiple comparisons.

This example describes a method for testing the immunogenicity of a live attenuated serotype 14 adenovirus in a mammal.

### Example 3

This example demonstrates a method of inducing an immune response against a live attenuated serotype 14 adenovirus in a mammal.

Mice were injected with either one or two administrations of 1×10⁹ pu of an E1-deleted serotype 14 adenovirus containing an expression cassette for a fragment of HIV envelope (gp140B) inserted into the deleted E1 region. Serum was taken at six weeks and evaluated in neutralization assays for both the DeWitt strain and new Ad14 wildtype virus, 1968T. For animals receiving a second (i.e., boost) administration of adenovirus, adenovirus was administered at six weeks, and serum was sampled four weeks later. Significant titers of neutralizing antibody were induced following a single administration of adenovirus, which were further increased upon a second administration of adenovirus.

These results strongly suggest that a live attenuated serotype 14 adenovirus that lacks a transgene will generate a high titer of neutralizing antibody reactive to both the DeWitt strain and 1968T strain of Ad14.

### Example 4

The example demonstrates a method of determining the safety of a live attenuated serotype 14 adenovirus in a mammal.

The tolerability of Ad14nr will be assessed in mice using body weight determinations conducted at high doses of Ad14nr. The experiments will be conducted as follows:

| | |
|---|---|
| Animals: | 10 female and 10 male Balb/c mice per group |
| Dose | 1 × 10¹⁰ pu Ad14nr, 1 × 10¹¹ pu Ad14nr, 1 × 10¹⁰ pu |
| Groups: | Ad5null, 1 × 10¹¹ pu Ad5null, and formulation buffer only |
| Routes: | Intramuscular and intravenous |
| Endpoints: | Daily body weight determinations for 21 days post injection. |

Preliminary biodistribution will be monitored at 21 days. Biodistribution will be conducted using a quantitative PCR (qPCR) assay specific for the deleted E1 region of Ad14nr and Ad5null. Tissues that will be monitored include liver, spleen, kidney, brain, gonads, heart, lungs, as well as the injection site. Analysis of these tissues will identify accumulation of adenovirus in tissues that are relevant for tolerability and may need to be considered during future toxicology studies. An Ad14nr qPCR assay will be developed and tested prior to initiation of the aforementioned biodistribution study.

Primer sets suitable for specific detection of the A14 genome will be similar to those used to analyze the E1 region deletion as described in Example 1. qPCR methods for genome quantification are routinely used in the art. The specificity, intra-assay precision, and accuracy of the qPCR assay used in the context of these experiments have been evaluated as part of a GLP study and subsequently submitted to the FDA in an ND filing (Althea Report Nos. J106-001, J106-002). This assay was found to be suitable in quantifying copy number of adenovirus vector constructs. For each qPCR run to qualify, the run must meet the following acceptance criteria to be considered valid: (1) the correlation coefficient of the standard curve (r²) must be ≧0.980, (2) the negative extraction control must be below the limit of detection, and (3) the no template control must show no amplification. The lower limit of detection of this assay was found to be 10 copies/sample and the lower limit of quantitation is 50 copies/sample.

The example describes a method of determining the safety of a live attenuated serotype 14 adenovirus in a mammal.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inducing an immune response against a serotype 14 adenovirus in a mammal, which method comprises administering to the mammal a live attenuated serotype 14 adenovirus, whereupon an immune response against a serotype 14 adenovirus is induced in the mammal.

2. The method of claim 1, wherein the live attenuated serotype 14 adenovirus requires complementation of, at most, the E1 region, the E2A region, and/or the E4 region of the adenoviral genome for propagation.

3. The method of claim 1, wherein the live attenuated serotype 14 adenovirus does not require complementation of the E2A region of the adenoviral genome for propagation.

4. The method of claim 1, wherein the live attenuated serotype 14 adenovirus requires complementation of the E1 region of the adenoviral genome for propagation.

5. The method of claim 1, wherein the live attenuated serotype 14 adenovirus comprises an adenoviral genome that lacks all or a portion of the E1 region.

6. The method of claim 1, wherein the live attenuated serotype 14 adenovirus requires complementation of the E4 region of the adenoviral genome for propagation.

7. The method of claim 6, wherein the live attenuated serotype 14 adenovirus comprises an adenoviral genome that lacks all or a portion of the E4 region.

8. The method of claim 1, wherein the live attenuated serotype 14 adenovirus comprises an adenoviral genome that lacks all or a portion of the E3 region.

9. The method of claim 1, wherein the live attenuated serotype 14 adenovirus does not comprise a heterologous nucleic acid sequence.

10. The method of claim 1, wherein the method comprises administering a priming composition to the mammal prior to administering live attenuated serotype 14 adenovirus to the mammal.

11. The method of claim 10, wherein the priming composition comprises a live attenuated serotype 14 adenovirus.

12. The method of claim 1, wherein the method comprises administering a boosting composition to the mammal after administering the live attenuated serotype 14 adenovirus to the mammal.

13. The method of claim 12, wherein the boosting composition comprises a live attenuated serotype 14 adenovirus.

14. The method of claim 1, wherein the mammal is a human.

15. A live attenuated serotype 14 adenovirus.

16. The live attenuated serotype 14 adenovirus of claim 15, wherein the live attenuated serotype 14 adenovirus requires complementation of, at most, the E1 region, the E2A region, and/or the E4 region of the adenoviral genome for propagation.

17. The live attenuated serotype 14 adenovirus of claim 15, wherein the live attenuated serotype 14 adenovirus does not require complementation of the E2A region of the adenoviral genome for propagation.

18. The live attenuated serotype 14 adenovirus of claim 15, wherein the live attenuated serotype 14 adenovirus requires complementation of the E1 region of the adenoviral genome for propagation.

19. The live attenuated serotype 14 adenovirus of claim 15, wherein the live attenuated serotype 14 adenovirus comprises an adenoviral genome that lacks all or a portion of the E1 region.

20. The live attenuated serotype 14 adenovirus of claim 15, wherein the live attenuated serotype 14 adenovirus requires complementation of the E4 region of the adenoviral genome for propagation.

21. The live attenuated serotype 14 adenovirus of claim 20, wherein the live attenuated serotype 14 adenovirus comprises an adenoviral genome that lacks all or a portion of the E4 region.

22. The live attenuated serotype 14 adenovirus of claim 15, wherein the live attenuated serotype 14 adenovirus comprises an adenoviral genome that lacks all or a portion of the E3 region.

23. The live attenuated serotype 14 adenovirus of claim 15, wherein the live attenuated serotype 14 adenovirus does not comprise a heterologous nucleic acid sequence.

24. A composition comprising the live attenuated serotype 14 adenovirus of claim 15 and a pharmaceutically-acceptable carrier.
